(19) United States
(12) Patent Application Publication
(10) Pub. No.: US 2009/0155363 A1
(43) Pub. Date: Jun. 18, 2009

(54) METHODS FOR ORAL ADMINISTRATION OF ACTIVE DRUGS

(76) Inventor: Todd Maibach, Menlo Park, CA (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 11/956,616

(22) Filed: Dec. 14, 2007

(51) Int. Cl. A61K 47/00; A61K 47/26; A61K 47/38; A61K 9/00; A61K 47/32; A61K 47/42

(52) U.S. Cl. 424/484; 514/777; 514/781; 514/773; 514/774; 514/778; 514/782; 514/779; 514/772.4; 514/772.5

(57) ABSTRACT

The present invention relates to methods that facilitate the oral administration of active drugs to a patient. Specifically, the methods of the present invention may utilize compositions comprising an active drug and a gelling agent that provides an easily consumable gel dosage form and the active drug is homogenously mixed within the gel.

### FIELD OF THE INVENTION

The present invention relates to methods for the facilitated oral administration of active drugs to a patient that has difficulty swallowing or chewing. Specifically, the methods comprise a drug delivery system in a gel dosage form and conveniently prepackaged for the oral administration of active drugs to a patient.

### BACKGROUND OF THE INVENTION

Delivery systems of over the counter or prescription drugs come in a variety of dosage forms. The majority of dosage forms come in a pill-like product such as in a capsule, tablet, caplet or gel cap. However, such pill-like dosage forms may be incompatible to many patients. In particular, such pills may be incompatible to patients such as the elderly or infants who have difficulty swallowing a whole pill or have difficulty chewing chewable pills. In order to overcome this common problem, hospitals or nursing homes perform various means to administer drugs to elderly patients or infants who cannot consume a pill. Perhaps the most common method is to break up or crush the pill and grind it or mix it into a more easily swallowable medium. Common swallowable mediums include foods such as applesauce or ice cream.

There are multiple problems with mixing the contents of a pill with food. First, it takes time and energy for a nurse or caregiver to break up a pill and mix or dissolve it into a more swallowable medium. Second, the mixing lacks proper quality control in order to make sure that the active drug is mixed evenly into the swallowable medium. Hence, if a patient only consumes half of the apple sauce or swallowable medium, it would be difficult for a nurse or physician to know how much of the active drug was actually administered to the patient. Moreover, even if the patient consumed the majority of the swallowable medium, the active drug may not have been properly mixed and may be heavily concentrated on the sides of the bowl or within the left over remains at the bottom of the howl. Third, the addition of bitter tasting pills may have an unpleasant and bitter effect on the taste of the swallowable medium. Fourth, the opening of the pill and introducing the active drug to a new medium may have a profound effect on the bioavailability or intended function of the drug. For example, research indicates that administration of the drug nizatidine mixed with foods such as applesauce or vegetable juice reduces the bioavailability approximately 30 to 40% relative to administration of a nizatidine capsule with water. Sullivan et al., 2(4) AM. J. THER. 275-278. (1995). Furthermore, when administering a drug in applesauce instead of in a time release capsule is believed to have an impact on the Cₘₐₓ of the drug. The method of administration of active drugs with other compounds in applesauce may also lead to adverse interactions with the drug before administration. For example, adding a drug to applesauce that is too warm can have deleterious effects on the active drug. Hence, the addition of the active drug to applesauce or other mediums may have unintentional, hut adverse effects on the active drug due to no proper quality control. There are therefore numerous problems with this common method currently in use. A need therefore exists for the oral delivery of an active drug to a patient that has difficulty swallowing or chewing beyond what is currently available.

### SUMMARY OF THE INVENTION

The present invention provides methods for utilizing compositions for oral drug administration to a patient. Specifically, the present invention provides methods for facilitated oral administration of an active drug to a patient comprising administering an active drug in a gel form to a patient, wherein the gel form comprises one or more gelling agents and an active drug, and the gel form comprises a homogenous mixture of the active drug.

In another embodiment of the present invention, the methods may include oral administration to a patient that has difficulty in swallowing pills.

In another embodiment of the present invention, the methods may include oral administration to an elderly patient.

In another embodiment of the present invention, the methods may include oral administration to a patient that is an infant and/or child.

In another embodiment of the present invention, the methods may utilize compositions wherein the gelling agent is in the form of a carbohydrate.

In another embodiment of the present invention, the methods may utilize a composition comprising a carbohydrate selected from one or more of the group consisting of aldohexoses, disaccharides, polysaccharides, ketohexoses, glucose, glucose polymers, dextrose, maltose, maltodextrins, mattotriose, lactose, galactose, sucrose, corn syrup, high fructose corn syrup, honey, maple syrup, molasses, brown rice syrup, beet sugar, cane sugar, sucanat, arabinose, ribose, xylose, fructose, levulose, psicose, sorbose, tagatose, sorbitol and combinations thereof.

In another embodiment of the present invention, the methods may utilize a composition comprising a carbohydrate selected from one or more of the group consisting of maltodextrin and fructose.

In another embodiment of the present invention, the methods may utilize a composition comprising a carbohydrate in the form of brown rice syrup.

In another embodiment of the present invention, the methods may utilize a composition comprising a gelling agent selected from one or more of the group consisting of protein, amino acids, pectin, agar, arabic gum, xanthan gum, tragacanth gum, karaya gum, ghatti gum, guar gum, gellan gum, locust bean gum, alginic acid, a pharmaceutically acceptable alginate salt, carrageenan, gelatin, dextrin, starches, corn starch, rice starch, wheat starch, potato starch, pueraria starch, tapioca starch, carboxymethyl starch, hydroxypropyl starch, hydroxyethyl starch, chemically cross-linked starch, celluloses, hydroxypropylmethyl cellulose, carboxymethyl cellulose, methyl cellulose, methylethyl cellulose, hydroxypropyl cellulose, crystalline cellulose, polyvinyl alcohol, polyvinylpyrrolidone, polyethylene glycol, mannans, and combinations thereof.

In another embodiment of the present invention, the methods may utilize a composition comprising an active drug that is an antacid or drug to treat peptic ulcer disease selected from one or more of the group consisting of proton pump inhibitors omeprazole, lansoprazole, pantoprazole; H2 receptor antagonists cimetidine, ranitidine, famotidine, nizatidine, carbamazepine; the prostaglandins misoprostol; antimicrobial agents amoxicillin, bismuth compounds, clarithromycin, metronidazole, and tetracycline.

In another embodiment of the present invention, the methods may utilize a composition comprising an antiparkinsons drug selected from one or more of the group consisting of amantadine, antimuscarinic agents, bromocriptine, carbidopa, deprenyl, levodopa, pramipexole, repinirole, tolcapone, benztropine, trihexyphenidyl, biperiden. bromocriptine, pergolide, pramipexole, ropinirole, cabergoline, apomorphine, lisuride, selegiline and rasagiline.

In another embodiment of the present invention, the methods may utilize a composition comprising antidepressants selected from one or more of the group consisting of amitriptline, amoxapine, desipramine, doxepin, imipramine, maprotiline, nortiptyline, prtriptyline, trimipramine, fluoxetine, flvoxamine, nefaxodone, paroxetine, sertraline, trazadone, venlafaxine, isocarboxazid, phenelxine, tranylcypromine and lithium salts.

In another embodiment of the present invention, the methods may utilize a composition comprising phosphate binders selected from one or more of the group consisting of aluminum hydroxide, sevelamer, lanthanum carbonate, calcium acetate and calcium carbonate.

In another embodiment of the present invention, the methods may utilize anti-psychotics selected from one or more of the group consisting of chlorpromazine, clozapine, fluphenazine, haloperidol, haloperidol decanoate, prochlorperazine, promethaxine, risperidone, thioridazine, thiothixene, olanzapine, aripiprazole, quetiapine, and paliperidone.

In another embodiment of the present invention, the methods may utilize a composition comprising antiepileptic drugs selected from one or more of the group consisting of carbamazepine, clonazepam, clorazepate, diazepam, ethosuximide, gabapentin, lamotrigine, phenobarbital, phenytoin, primidone, tiagabine, topiramate, valproic acid, divalproex sodium (valproate semisodium) and vigabatrin.

In another embodiment of the present invention, the methods may utilize a composition comprising anti-hypertensives and other cardiovascular drugs selected from one or more of the group consisting of: diuretics acetazolamide, bumetanide, ethacrylic acid, spironolactone, furosemide, torsemide, cholorthiaxide, chlorthalidone, hydrochlorothiazide, indapamide, metolazone, amiloride, spironolactone, triamterene, and urea; Ace inhibitors benazepril, captopril, enalapril, fosinopril, lisinopril, moexipril, quinapril and ramipril; Beta blockers acebutolol, esmolol, pindolol, atenolol, labetalol, metoprolol, nadolol, propranolol and timolol; Angiotensin II antagonists valsartan, losartan, irbesartan, olmesartan, telmisartan, eprosartan, and candesartan; HMG-CoA reductase inhibitors (statins) atorvastatin, simvastatin, pravastatin, atorvastatin, fluvastatin cerivastatin, mevastatin and pitavastatin; and the nonsteroidal anti-inflammatory drug aspirin.

In another embodiment of the present invention, the methods may utilize a composition comprising antiarrhythmic drugs selected from one or more of the group consisting of disopyramide, flecainide, lidocaine, mexiletine, procainamide, propafenone, quinidine, tocainide, esmolol, metoprolol, pindolol, propranolol, amiodarone, bretylium, sotalol, diltiazem, verapamil, adenosine and digoxin.

In another embodiment of the present invention, the methods may utilize a composition comprising hormonal, endocrine or drugs for the thyroid selected from one or more of the group consisting of iodide, levothyroxine, methimazole, propylthiouracil, thyroxine, triiodothyronine, homones of the posterior pituitary, desmopressin, exytocin and vasopressin, hypothalamic and anterior pituitary hormones, corticotropin, gonadotropin-releasing hormone, growth, hormone releasing hormone, sermorelin, luteinizing hormone-releasing hormone, leruprolide, goserelin, nafarelin, histrelin, somatostatin, octrotide, somatotropin and somatrem.

In another embodiment of the present invention, the methods may utilize a composition comprising antidiarreheal, stool softener or laxative drugs selected from one or more of the group consisting of docusate, mineral oil, castor oil, senna, aloe, phenolphthalein, bisacodyl, hydrophilic colloids, methylcellulose, psyllium seeds, bran magnesium sulfate, magnesium hydroxide, polyethylene glycol, lactulose, sorbitol sodium phosphate, diphenoxylate, loperamide, kaolin, pectin, activated attapulgite, indomethacin and bismuth subsalicylate.

In another embodiment of the present invention, the methods may utilize a composition comprising a drug for treatment of urinary incontinence selected from one or more of the group consisting of tolterodine, oxybutynin, propantheline, hyoscyamine, imipramine, flavoxate, solifenacin, dicyclomine and darifenacin.

In another embodiment of the present invention, the methods may utilize a composition comprising an analgesic/antipyretic selected from one or more of the group consisting of acetaminophen, buprenorphine, butorphanol, codeine, dextropropoxyphene, dihydrocodeine, fentanyl, hydrocodone, hydromorphone, ketobemidone, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, aspirin, choline salicylate magnesium salicylate, sodium salicylate, ibuprofen, naproxen and ketoprofen.

In another embodiment of the present invention, the methods may utilize a composition comprising a pediatric drug.

In another embodiment of the present invention, the methods may utilize a composition comprising a pediatric drug selected from one or more of the group consisting of epinephrine, corticosteroids, pyrimethamine, sulfadiazene, leucovorin, penicillin, erythromycin, tetracycline, acetaminophen, ibuprofen, imipramine, methylphenidate, dextroamphetamine, clonidine, piracetam, hormones, ceftriaxone, metronidazole, clindamycin, cefoxitin, ampicillin, ampicillin-sulbactam, clarithromycin, azithromycin, aspirin, codeine, acyclovir, valacyclovir, famciclovir, measles immune globulin, anticonvulsants, NSAIDs, ribavirin, loperamide, metoclopramide, mineral oil, milk of magnesia, vincristine, cyclophosphamide, doxorubicin, etoposide and cisplatin.

In another embodiment of the present invention, the methods may utilize a composition comprising an antibacterial selected in an one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin.

In another embodiment of the present invention, the methods may utilize a composition comprising a preservative. The methods may utilize a composition comprising a preservative selected from one or more of the group consisting of sodium benzoate, potassium sorbate, sorbic acid, sodium sorbate, citric acid, and calcium sorbate.

In another embodiment of the present invention, the methods may utilize a composition comprising an electrolyte/mineral selected from one or more of the group consisting of sodium, potassium and magnesium.

In another embodiment of the present invention, the methods may utilize a composition comprising vitamins nutrients and/or mineral selected from one of more of the group consisting of vitamin A, vitamin D, vitamin K, vitamin E, vitamin C, vitamin B₁, vitamin B₂, vitamin B₃, vitamin B₅, vitamin B₆, vitamin B₇, vitamin B₉ and vitamin B₁₂, pyridoxal, pyridoxamine, pantothenic acid, folic acid, sodium, potassium, chloride, magnesium, manganese, and Omega-3 fatty acids.

In another embodiment of the present invention, the methods may utilize a composition comprising fiber. In another embodiment of the present invention, the methods may utilize a composition comprising fiber for administration to a patient in need of fiber.

In another embodiment of the present invention, the methods may utilize a composition comprising ingredients containing extra nutrients or calories. The extra nutrients or calories may compose protein, amino acids or additional carbohydrates. In another embodiment of the present invention, the methods may utilize compositions comprising nutrients or calories for administration to a patient in need of extra calories.

In another embodiment of the present invention, the methods may utilize a composition comprising a range of about 150 large calories per 32 g to about 50 large calories per 32 g.

In another embodiment of the present invention, the methods may utilize a composition comprising about 100 large calories per 32 g.

In another embodiment of the present invention, the methods may utilize a composition comprising a masking flavor. The masking flavor may be a natural or artificial flavor selected from one or more of the group consisting of the flavors apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

In another embodiment of the present invention, the methods may utilize a composition comprising a natural color, natural dye, artificial color or natural dye.

In another embodiment of the present invention, the methods may utilize a composition comprising a range of viscosity of about 1 cps to about 100,000 cps. In another embodiment of the present invention, the methods may utilize a composition comprising a range of viscosity of about 1 cps to about 10,000 cps. In another embodiment of the present invention, the methods may utilize a composition comprising a range of viscosity of about 10 cps to about 250 cps.

In another embodiment of the present invention, the methods may utilize a composition comprising a range of about 90% to about 30% maltodextrin by weight.

In another embodiment of the present invention, the methods may utilize a composition comprising a range of about 50% to about 5% fructose by weight.

In another embodiment of the present invention, the methods may utilize a composition comprising about 85% complex carbohydrates from maltodextrin and 15% simple carbohydrates from fructose.

In another embodiment of the present invention, the methods may utilize a composition comprising about 70% carbohydrates from maltodextrin and about 30% carbohydrates from fructose.

In another embodiment of the present invention, the methods may utilize a composition comprising maltodextrin, fructose, water, sodium citrate, potassium citrate, natural or artificial flavors and sodium benzoate.

In another embodiment of the present invention, the methods may utilize a composition comprising brown rice syrup, natural flavors, sea salt, potassium citrate, citric acid, and magnesium oxide.

In another embodiment of the present invention, the methods may utilize a composition comprising by weight about 68% maltodextrin, about 19% water, about 9% fructose, about 0.05% sodium benzoate, about 1.3% sodium citrate, about 0.4% potassium citrate, about 0.3% natural and/or artificial flavors, about 0.05% potassium sorbate and the appropriate dosage amount of the active drug.

In another embodiment of the present invention, the methods may utilize a composition packaged per unit dose. The total weight per unit dose or serving size may be in the range of about 1 g to about 48 g. The total weight per unit dose or service size may be in the amount of about 32 g. The total volume per unit dose or serving volume may be in the range of about 1 ml to about 10 mL.

In another embodiment of the present invention, the methods may utilize a composition comprising packaging designed to fit on a nursing home medication cart.

In another embodiment of the present invention, the methods may utilize a composition comprising packaging designed to fit in the patient's individual tray.

In another embodiment of the present invention, the methods may utilize a composition comprising packaging designed for a person to administer the composition comprising an active drug to a patient.

In another embodiment of the present invention, the methods may utilize a composition comprising packaging in the form of one or more of the group consisting of a bottle, a can, a sealed cup, a box, a syringe, a foil or plastic pouch and a tube.

In another embodiment of the present invention, the methods may utilize a composition for oral administration to a patient by deformation of the packaging.

In another embodiment of the present invention, the methods may utilize a composition comprising packaging designed for administration to a patient with a straw.

In another embodiment of the present invention, the methods may utilize a composition comprising packaging selected from one or more of the group consisting of a tear-off top, a means for a straw to be inserted, a flip-up top, and a screw-off top.

In another embodiment of the present invention, the methods may utilize a composition comprising packaging that allow for dosages of active drugs that are too large for pill or filmstrip dosage forms.

In another embodiment of the present invention, the methods may utilize a composition substantially free of an added active drug. In another embodiment of the present invention, the methods may utilize a composition substantially free of an added active drug to allow the active drug to be added by a pharmacist.

In another embodiment of the present invention, the methods may utilize a composition that is co-administered, with another active drug.

In another embodiment of the present invention, the methods may utilize a composition substantially free of other added active ingredients.

### DETAILED DESCRIPTION OF THE INVENTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a”, “an,” and “the” include the plural reference unless the context clearly dictates otherwise. Thus, for example, a reference to “a carbohydrate” is a reference to one or more carbohydrates and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal, including mammals.

The term “administrable” defines a composition that is able to be given, to a patient. Likewise, “administering” refers to the act of giving a composition to a patient or otherwise making such composition available to a patient or the patient taking a composition.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than, the active ingredient.

By “active ingredient” or “active drug” then, is meant any compound intended to furnish pharmacological activity or other direct effect in the diagnosis, cure, mitigation, treatment and/or prevention of a condition. See 21 C.F.R. 210.3(b)(7). Further, “active ingredients” or “active drugs” include those compounds of the composition that may undergo chemical change during the manufacture of the composition and be present in the final composition in a modified form intended to furnish an activity or effect. Id.

The term “substantially free.” as used herein, means free from therapeutically effective amounts of compounds when administered in suggested dosages, but may include trace amounts of compounds in non-therapeutically effective amounts.

The term “homogenous” in the present invention means the state where the referenced component of the composition is uniformly dispersed and there is no uneven distribution of the component in the composition.

The term “dosage form,” as used herein, is the form in which the dose is to be administered to the patient. The drug is generally administered as part of a formulation that includes nonmedical agents, referred to as pharmaceutical ingredients. The dosage form has unique physical and pharmaceutical characteristics. Dosage forms may be solid, semisolid, liquid or gaseous. Solid forms include, but are not limited to capsules, tablets, caplets, gel caplets (gelcap), lozenges, wafers etc. Liquid dosage forms include, but are not limited to, elixirs, injectable solutions, and intravenous solutions. Gaseous forms include vapors, inhalants, and the like.

Dosage forms may also comprise unique physical characteristics that may include both a liquid and solid dosage form. For example, semisolid dosage forms which include, but are not limited to, goos, gels and syrups may be referred to as a solid form or a liquid form. Specifically, a “gel form” or “gel dosage form” refers to a viscous fluid, semi-liquid or semisolid form.

Delivery systems of over the counter or prescription drugs come in a variety of shapes and pills, such as in a capsule, tablet, caplet, or gel caplet (gelcap). However, due to consumer perception and patient preference, certain dosage forms have particular advantages over others. Until the 1980's, capsules were the preferred form for the delivery of active drugs. Capsules are a dosage form in which the drug is housed within two halves of gelatin shells that are banded together. Due to the ability of taking apart the capsule gelatin shell halves and then resealing the halves, tampering of the capsule became a major problem. For this reason, the pharmaceutical industry withdrew many of their capsule products from the market, often replacing them with tablets or caplets.

Tablets and caplets are compressed solid pills where the dosage forms are cylindrical or the oblong shape similar to capsules. Although both dosage forms are presently used in the market, tablets and caplets have not reached the same level of consumer acceptance that capsules once had. Consumer surveys suggest that a dosage form with the like appearance of a gelatin coated capsule is easier to swallow and that the drug contained in the capsule form will be more effective.

The gelcap is a drug product that may itself encompass many forms. For example, the gelcap may contain a filler containing the active drug in a liquid form that is encapsulated in a gelatin capsule like shell. Also, a gelcap can contain the active drug in a solid form, which has the shape of a caplet and a gelatin coating or covering to create the appearance and therefore, the consumer acceptability of the capsule.

A gelcap may also contain the active drug in a liquid form. Concentrated liquid compositions are well suited for encapsulation within a soft gelatin shell. The active drug contained in the liquid form may provide advantages by dispersing the drug to the active site. For example, the active drug does not first have to dissolve in the gastrointestinal tract, thereby facilitating absorption of the pharmacologically active substance. See, for example, U.S. Pat. No 6,689,382, which is expressly incorporated by reference herein. Other formulations take advantage of the liquid form by creating a sustained release gelatin capsule, thereby permitting the delivery of the drug in a controlled fashion. See, for example, U.S. Pat Nos. 5, 324,280 and 6,929,803, which are expressly incorporated by reference herein.

However, none of the above mentioned dosage forms solve the problem for a patient that has a difficult time swallowing whole pills and/or chewing pills. This problem is particularly evident in nursing homes, where elderly patients are prescribed drugs that are available only in a pill dosage form. In order to overcome this common problem, hospitals or nursing homes perform various means to administer drugs to elderly patients or infants who cannot swallow a whole pill. Perhaps the most common method is to break up or crush the pill and grind it or mix it into a more easily swallowable medium. Common swallowable mediums include foods such as applesauce or ice cream.

There are however multiple problems with mixing the contents of a pill into a food for the purpose of patient compliance. First, it takes time and energy for a nurse or caregiver to break up a pill and mix or dissolve it into a more swallowable medium. Second, the mixing lacks proper quality control in order to make sure that the active drug is mixed evenly into the swallowable medium. Hence, if a patient only consumes half of the apple sauce or swallowable medium, it would he difficult for a nurse or physician to know how much of the active drug was actually administered to the patient. Moreover, even if the patient consumed the majority of the swallowable medium, the active drug may not have been properly mixed and maybe heavily concentrated on the sides of the bowl or the very last remains of the medium at the bottom of the bowl. Third, the addition of bitter tasting pills may have an unpleasant and bitter effect on the taste of the applesauce. Fourth, the opening of the pill and introducing the active drug to a new medium may have a profound effect on the bioavailability or intended function of the drug. For example, research indicates that administration of the drug nizatidine mixed with foods such as apple sauce or vegetable juice reduces the bioavailability approximately 30 to 40% relative to administration of a nizatidine capsule with water. Sullivan et al., 2(4) AM. J. THER. 275-278. (1995). Furthermore, administering a drug in applesauce instead of in a time release capsule is believed to have an impact on the Cₘₐₓ of the drug. The method of administration of active drugs with other compounds in applesauce may also lead to adverse interactions with the drug before administration. For example, adding a drug to applesauce that is too warm can have deleterious effects on the active drug. Hence, the addition of the active drug to applesauce or other mediums may have unintentional, but adverse effects on the active drug due to no proper quality control.

The industry has provided other dosage forms to administer drugs orally that do not require the chewing of pills or swallowing of whole pills. Some investigators have suggested that it may be possible to administer medication through the buccal mucosa of the cheek pouch or by sublingual administration. See U.S. Pat. No. 4,671,953. Generally the drugs which are administered by any of these methods have an unpleasant taste. As a result, in order to allow for buccal or sublingual administration through the oral mucosal tissues, it is also necessary to incorporate the drug into some type of pleasant tasting mass, such as a “candy” matrix.

For effective application of the drug, a candy product may contain the drug uniformly distributed throughout in order to ensure uniform levels of medication. Alternatively, for some applications, varying concentrations within known and controlled ranges may be desired to vary the rate of drug administration. Difficulties are encountered in attempting to blend solid drugs in a uniform or otherwise carefully controlled manner. Many drugs are insoluble, or only partially soluble in one or more of the ingredients of the hard candy base. Thus, the resultant product is often found to be lacking in uniform or controlled distribution of the drug. Moreover, sublingual tablets also experience issues related to inter-tablet migration of an active drug, similar to the sustained-release tablet methodology, which can produce a high degree of weight and close variation between tablets.

Furthermore, many presently available medicated candy lozenges tend to crumble when placed in the mouth. As a result, uniform release of the drug into the mucosal tissues does not take place. Rather, the crumbled lozenge is mostly chewed, and swallowed. Thus, it will be appreciated that candy lozenges have very definite limitations for use in the administration of a drug through the oral mucosal tissues. As a result, candy or lozenge dosage forms are not a desirable alternative to the pill dosage form for patients that have difficulty chewing or swallowing.

In another example, edible films may be used for oral administration of drugs. Edible films or film strips are biodegradable thin films comprising an active drug that are adapted to rapidly dissolve in the patient's mouth. Dissolution of the film therefore releases the active drug which may be absorbed in the oral mucosal tissue of the patient. However, filmstrips are also limited. For example, edible films are limited to smaller dosages, approximately 30 mg of an active drug. Hence, delivery of many active drugs in the required dosage is not possible in the edible film dosage form.

Because of the many issues of delivering an active drug to a patient that has difficulty swallowing or chewing, it would be beneficial to have a drug delivery system that could allow easy oral administration, but also provide convenience, proper quality control not currently available, and the ability to have a flavorful and overall patient friendly delivery system.

A drug delivery system where the active drug is administered within a gel medium could be a possible and desirable solution. A gel or semi-soluble drug delivery medium would, allow the patient to consume the ingredients, including the active drug, without having to chew or ingest large or difficult-to-swallow pills. Moreover, the addition of various flavors to the gel-like medium would allow for a flavorful and non-bitter taste when consuming the active drug and other included contents.

A gel medium/gel form that is currently in use today is in the sports drink/energy gel market. Energy gel products currently in the market provide a convenient means for an athlete during or after exercise to replenish electrolytes and provide quick energy in the form of carbohydrates. The advantage of the gel form is that it allows for ingredients to be highly concentrated but still be consumed in a convenient way that does not require chewing or swallowing of solids. For example, an athlete such as a biker or runner can maintain a competitive pace during a race, while consuming an energy gel from a tube or easy-to-rip foil pouch. The athlete can consume the energy gel without stopping or being substantially slowed down by the chewing and/or swallowing that is required from an energy bar. Moreover, because the gel form is in a semi-solid or viscous state, it can still pack highly concentrated amounts of ingredients such as carbohydrates that are beneficial for athletes to obtain quick energy. Unlike with an energy drink, an athlete does not have to consume or swallow large amount of contents to replenish. Hence, the athlete consuming a gel does not have to be slowed down during energy consumption for as long as an athlete consuming a relatively diluted energy drink. The energy gel therefore provides two advantages for easier consumption: 1) no chewing or swallowing of difficult to swallow-solids and 2) less total volumes to swallow/consume the necessary or intended ingredients.

Such a delivery method could therefore also be desirable and beneficial for administering an active drug to an elderly patient who has difficulty chewing and/or swallowing, or a child/infant that prefers to consume as little of an amount of a medicine as possible.

Moreover, a product with an active drug in a gel dosage form could provide a solution to the multiple problems that exist today with nursing homes mixing the contents of a pill with food. If such a product was provided already packaged per a unit dose to a nursing home, it would save the nurse or caregiver time from having to prepare a drug incorporated food for each patient. Moreover, such a product would provide the proper quality control that is lacking in a nursing home or home of the caregiver. Proper quality control could more accurately verify that the active drug is uniformly mixed within the food contents and that the contents in the food do not have an adverse effect on the functionality of the active drug. Another advantage would be the use of various forms of packaging or methods to administer the drug to a patient so that only a minimal or insignificant amount of gel residue has not been consumed. For example, the gel dosage form could be contained within a non-adherent film pouch that is easily deformed by the patient or administrator of the drug. When the gel dosage form is administered to the patient, only a limited and/or statistically insignificant amount of gel residue that contains the active drug remains unconsumed. Therefore, the availability of such a product allows the physician and/or nurse to more accurately determine the drug dosages administered to the patient.

In a specific embodiment, the methods of the present invention include the facilitated oral administration of an active drug to a patient wherein the composition is in a gel form and comprises one or more gelling agents and one or more active drugs and the gel form comprises a homogenous mixture of the active drug, in another specific embodiment, the methods of the present invention include oral administration to patients that have difficulty in swallowing pills. Specifically, such patients may include, but are not limited to, the elderly, children and/or infants.

In a specific embodiment of the invention, the methods may utilize compositions comprising one or more gelling agents. Herein, a gelling agent refers to any ingredient that assists in creating a viscous liquid, semi-liquid, or semisolid, medium. The gel ling agent, therefore assists in the formation of a gel dosage form, which may allow a patient to orally take or to be administered an active drug without the action of chewing or the swallowing of a pill. The gelling agent may be a dry ingredient such as a carbohydrate. In order to create a gel dosage form from dry ingredients, a liquid may be added. Depending on patient preference, other added ingredients, and preferred viscosity, the amount of liquid added may vary.

The gelling agent may also be a liquid ingredient. For example, liquids such as brown rice syrup, which comprise carbohydrates in a syrup form, may be used as a gelling agent. The amount of liquid gelling agent added to dry ingredients may also vary depending on the desired viscosity of the composition. In a specific embodiment, water is the liquid used in the manufacturing process to turn the dry ingredients into a gel form. However, fruit juice, such as apple juice may be used in addition to, or in place of water.

Carbohydrates, such as maltodextrin added with a liquid creates a gel form that may provide a consistency that is more easily consumed by a patient. In a specific embodiment, the methods of the present invention may utilize a gelling agent that is a carbohydrate. Maltrodoxin is a popular carbohydrate and gelling agent in the energy gel industry. In a specific embodiment, the gelling agent may include maltodextrin.

In another specific embodiment of the present invention the gelling agent may be a carbohydrate selected from one or more of the group consisting of aldohexoses, disaccharides, polysaccharides, ketohexoses, glucose, glucose polymers, dextrose, maltose, maltotriose, lactose, galactose, sucrose, corn syrup, high fructose corn syrup, honey, maple syrup, molasses, brown rice syrup, beet sugar, cane sugar, sucanat, arabinose, ribose, xylose, fructose, levulose, psicose, sorbose, tagatose, sorbitol and combinations thereof.

In a specific embodiment, the gelling agent may include fructose. In a specific embodiment, the gelling agent may be in the form of brown rice syrup. The methods of the present invention may utilize more than one carbohydrate as a gelling agent. For example, the methods of the present invention may utilize the gelling agents maltodextrin and fructose.

The methods may also utilize compositions that have various ratios or percentages of one or more carbohydrates. The carbohydrates may include simple carbohydrates and/or complex carbohydrates. Complex carbohydrates include maltodextrin and other polysaccharides, including long chain polysaccharides. The methods may utilize compositions that are 100% complex carbohydrates. In a specific embodiment, the methods may utilize compositions that are by weight a range of about 90% to about. 30% maltodextrin. In a specific embodiment, the methods may utilize compositions that are at least 50% simple carbohydrates. In a specific embodiment, the methods may utilize compositions that are by weight a range of about 50% to about 5% fructose. In another specific embodiment, the methods may utilize compositions that are by weight the amount of about 85% complex carbohydrates from maltodextrin and 15% simple carbohydrates from fructose. In another specific embodiment, the methods may utilize compositions that are about 70% complex carbohydrates from maltodextrin and 30% simple carbohydrates from fructose.

The gelling agent may also be nutrients and may include protein and/or amino acids. In another specific embodiment, the methods may utilize the gelling agent selected from one of more of the group consisting of pectin, agar, arable gum, xanthan gum, tragacanth gum, karaya gum, ghatti gum, guar gum, gellan gum, locust bean gum, alginic acid, a pharmaceutically acceptable alginate salt, carrageenan, gelatin, dextrin, starches, corn starch, rice starch, wheat starch, potato starch, pueraria starch, tapioca starch, carboxymethyl starch, hydroxypropyl starch, hydroxyethyl starch, chemically cross-linked starch, celluloses, hydroxypropylmethyl cellulose, carboxymethyl cellulose, methyl cellulose, methylethyl cellulose, hydroxypropyl cellulose, crystalline cellulose, polyvinyl alcohol, polyvinylpyrrolidone, polyethylene glycol, mannans, and combinations thereof. The amount of liquid added, if needed, may vary depending on the gelling agent and the desired viscosity of the composition.

Active drugs may be added to the compositions for oral administration to a patient. The gel dosage form permits the addition of solid or liquid active drugs. The methods may utilize compositions comprising active drugs for elderly patients that have difficulty swallowing pills. For example the active drug may be an anti-hypertensive drug such as a diuretic, an ace inhibitor, a beta blocker, an angiotensin II antagonist, a HMG-CoA reductase inhibitor and an nonsteroidal anti-inflammatory. In a specific embodiment of the present invention, the methods may utilize compositions comprising active drugs selected from one or more of the group consisting of: diuretics acetazolamide, bumetanide, ethacrylic acid, spironolactone, furosemide, torsemide, cholorthiaxide, chlorthalidone, hydrochlorothiazide, indapamide, metolazone, amiloride, spironolactone, triamterene, and urea; Ace inhibitors benazepril, capropril, enalapril, fosinopril, lisinopril, moexipril, quinapril and ramipril; Beta blockers acebutolol, esmolol, pindolol, atenolol, labetalol, metoprolol, nadolol, propranolol and timolol; Angiotensin II antagonists valsartan, losartan, irbesartan, olmesartan, telmisartan, eprosartan, and candesartan; HMG-CoA reductase inhibitors (statins) atorvastatin, simvastatin, pravastatin, atorvastatin, fluvastatin cerivastatin, mevasiatin and pravastatin; and the nonsteroidal anti-inflammatory drug aspirin.

In another specific embodiment of the present invention, the methods may utilize a composition comprising an active drug that is an antacid or drug to treat peptic ulcer disease selected from one or more of the group consisting of proton pump inhibitors omeprazole, lansoprazole, pantoprazole; H2 receptor antagonists cimetidine, ranitidine, famotidine, nizatidine, carbamazepine; the prostaglandins misoprostol; antimicrobial agents amoxicillin, bismuth compounds, clarithromycin, metronidazole, and tetracycline.

In another specific embodiment of the present invention, the methods may utilize a composition comprising an antiparkinsons drug selected from one or more of the group consisting of amantadine, antimuscarinic agents, bromocriptine, carbidopa, deprenyl, levodopa, pramipexole, repinirole, tolcapone, benztropine, trihexyphenidyl, biperiden, bromocriptine, pergolide, pramipexole, ropinirole, cabergoline, apomorphine, lisuride, selegiline and rasagiline.

In another specific embodiment of the present invention, the methods may utilize a composition comprising antidepressants selected from one or more of the group consisting of amitriptline, amoxapine, desipramine, doxepin, imipramine, maprotiline, nortiptyline, prtriptyline, trimipramine, fluoxetine, flvoxamine, nefaxodone, paroxetine, sertraline, trazadone, venlafaxine, isocarboxazid, phenelxine, tranylcypromine and lithium salts.

In another specific embodiment of the present invention, the methods may utilize a composition comprising phosphate binders selected from one or more of the group consisting of aluminum hydroxide, sevelamer, lanthanum carbonate, calcium acetate and calcium carbonate.

In another specific embodiment of the present invention, the methods may utilize antipsychotics selected from one or more of the group consisting of chlorpromazine, clozapine, fluphenazine, haloperidol, haloperidol decanoate, prochlorperazine, promethaxine, risperidone, thioridazine, thiothixene, olanzapine, aripiprazole, quetiapine, and paliperidone.

In another specific embodiment of the present invention, the methods may utilize a composition comprising and epileptic drugs selected from one or more of the group consisting of carbamazepine, clonazepam, clorazepate, diazepam, ethosuximide, gabapentin, lamotrigine, phenobarbital, phenytoin, primidone, tiagabine, topiramate, valproic acid, divalproex sodium (valproate semisodium) and vigabatrin.

In another specific embodiment of the present invention, the methods may utilize a composition, comprising antiarrhythmic drugs selected from one or more of the group consisting of disopyramide, flecainide, lidocaine, mexiletine, procainamide, propafenone, quinidine, tocainide, esmolol, metoprolol, pindolol, propranolol, amiodarone, bretylium, sotalol, diltiazem, verapamil, adenosine and digoxin.

In another specific embodiment of the present invention, the methods may utilize a composition comprising hormonal, endocrine or drugs for the thyroid selected from one or more of the group consisting of iodide, levomyroxine, methimazole, propylthiouracil, thyroxine, triiodothyronine, homones of the posterior pituitary, desmopressin, oxytocin and vasopressin, hypothalamic and anterior pituitary hormones, corticotropin, gonadotropin-releasing hormone, growth hormone releasing hormone, sermorelin, luteinizing hormone-releasing hormone, leruprolide, goserelin, nafarelin, histrelin, somatostatin, octrotide, somatotropin and somatrem.

In another specific embodiment of the present invention, the methods may utilize a composition comprising antidiarreheal, stool softener or laxative drugs selected from one or more of the group consisting of docusate, mineral oil, castor oil, senna, aloe, phenolphthalein, bisacodyl, hydrophilic colloids, methylcellulose, psyllium seeds, bran magnesium sulfate, magnesium hydroxide, polyethylene glycol, lactulose, sorbitol sodium phosphate, diphenoxylate, loperamide, kaolin, pectin, activated attapulgite, indomethacin and bismuth subsalicylate.

In another specific embodiment of the present invention, the methods may utilize a composition comprising a drug for treatment of urinary incontinence selected from one or more of the group consisting of tolterodine, oxybutynin, propantheline, hyoscyamine, imipramine, flavoxate, solifenacin, dicyclomine and darifenacin.

In another specific embodiment of the present invention, the methods may utilize a composition comprising an analgesic/antipyretic selected from one or more of the group consisting of acetaminophen, buprenorphine, butorphanol, codeine, dextropropoxyphene, dihydrocodeine, fentanyl, hydrocodone, hydromorphone, ketobemidone, nalbuphine, oxycodone, oxymorphone, pentazocine, pethidine, tramadol, acetylsahcylic acid, diflunisal, ethenzamide, ammophenazone, metamizole, phenazone, phenacetin, ziconotide, tetrahydrocannabinol, aspirin, choline salicylate magnesium salicylate, sodium salicylate, ibuprofen, naproxen, and ketoprofen.

In another specific embodiment of the present invention, the methods may utilize a composition comprising an antibacterial selected from one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethyisuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isomazid; josamycin; kanamycin sulfate; kitasamycin; levofuraltadone; levopropylcillin potassium; lexithromycim; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpiciliin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virgiamycin and zorbamycin.

In another specific embodiment of the present invention, the methods may utilize a composition comprising an active drug that is used as a pediatric drug. The pediatric drug may be any drug that is used for adults, but also for children and/or infants. The pediatric drug may also be any drug that is predominantly administered to children and/or infants.

In another embodiment of the present invention, the methods may utilize a composition comprising a pediatric drug selected from one or more of the group consisting of epinephrine, corticosteroids, pyrimethamine, sulfadiazine, leucovorin, penicillin, erythromycin, tetracycline, acetaminophen, ibuprofen, imipramine, methylphenidate, dextroamphetamine, clonidine, piracetam, hormones, ceftriaxone, metronidazole, clindamycin, cefoxitin, ampicillin, ampicillin-sulbactam, clarithromycin, azithromycin, aspirin, codeine, acyclovir, valacyclovir, famciclovir, measles immune globulin, anticonvulsants, NSAIDs, ribavirin, loperamide, metoclopramide, mineral oil, milk of magnesia, vincristine, cyclophosphamide, doxorubicin, etoposide and cisplatin.

One or more preservatives may be utilized in the present invention. For example, the preservative may include, but is not limited to sodium benzoate, potassium sorbate, sodium sorbate, citric acid, sorbic acid, and calcium sorbate. The amount may vary depending on other ingredients, but may range from about 0.0% to about 1.5% by weight; about 0.1% to about 0.5%; and about 0.1% to about 1.0%. In a specific embodiment, the methods of the invention may utilize compositions with about 0.05% sodium benzoate. In a specific embodiment, the methods of the invention, may utilize compositions with about 0.05% potassium sorbate. In another specific embodiment, the methods of the invention may utilize compositions with about 1% citric acid.

The methods may utilize a composition wherein one or more nutrients, vitamins and/or minerals may be added. For example, one or more electrolytes/minerals may be added to the composition. In a specific embodiment of the present invention, the methods may utilize a composition comprising an electrolyte/mineral selected from one or more of the group consisting of sodium, potassium and magnesium. Specifically, sodium citrate and potassium citrate may be used. The total amount of the electrolytes may be from about 0.1% to about 5% by weight of the gelled composition. In a specific embodiment of the present invention, the methods may utilize a composition comprising fiber. In another specific embodiment of the invention, the methods may utilize compositions composing one of more vitamins or nutrients of the group consisting of vitamin A, vitamin D, vitamin K, vitamin E, vitamin C, vitamin B₁, vitamin B₂, vitamin B₃, vitamin B₅, vitamin B₆, vitamin B₇, vitamin B₉ and vitamin B₁₂, pyridoxal, pyridoxamine, pantothenic acid, folic acid, sodium, potassium, chloride, magnesium, manganese, and Omega-3 fatty acids.

The nutrient may be added where the patient is need of a particular nutrient or vitamin. For example, fiber may be added to the composition if the patient is need of fiber. The nutrient may be added where the patient is in need of extra calories. The extra source of calories may come from, but are not limited to protein, which includes amino acids, or a high concentration of carbohydrates.

The source of protein can be a protein fraction derived from any vegetable or animal source. Examples include whey protein, soy protein, rice protein, pea protein, beef protein, chicken protein, egg protein, and fish protein. Protein sources are available commercially. The amino acids may include, but are not limited, to natural, unessential and/or essential amino acids. In another specific embodiment of the invention, the methods may utilize compositions comprising amino acids selected from one or more of the group consisting of proline, phenylalanine, methionine, threonine, tryptophan, histidine, isoleucine, leucine, asparagine, aspartic acid, glutamic acid, glutamine, serine, tyrosine, valine, lysine, alanine, glycine, tryptophan, cysteine, trimethyl glycine (TMG), L taurine, L-carnitine and acetyl-L-carnitine.

In a specific embodiment of the invention, the methods may utilize compositions comprising in the range of about 150 large calories per 32 g to about 50 large calories per 32 g. In a specific embodiment of the invention, the methods may utilize compositions comprising in the amount of about 100 large calories per 32 g.

The methods may utilize a composition wherein, one or more masking flavors may be added. The flavors may be natural or artificial and may vary in type, intensity, concentration, consumer acceptance, etc. The flavors may include, but are not limited to apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

The methods may utilize a composition wherein one or more natural colors, natural dyes, artificial colors or artificial dyes may he added. The colors or dyes may vary in type, intensity, concentration, consumer acceptance, etc.

In another specific embodiment of the invention, the methods may utilize compositions that are substantially free of the added active drug. The compositions may be free of the active drug for later additions of a drug of choice by a pharmacist. Nursing homes, hospitals or the like can therefore utilize the compositions in a flexible manner, wherein an active drug of choice may be added, and/or a preferred dosage amount of the active drug may be added. In another specific embodiment of the invention, the methods may utilize compositions that comprise more than one active drug. The methods may also utilize compositions that are co-administered with another active drug.

In another specific embodiment of the invention, the methods may utilize compositions comprising various viscosities. The viscosity of the composition may vary depending on packaging, included ingredients and/or patient/consumer preference. In another specific embodiment of the invention, the methods may utilize compositions comprising a viscosity in the range of about 1 centipoise (cps) to about 100,000 cps. In another specific embodiment of the invention, the methods may utilize compositions comprising a viscosity in the range of about 1 cps to about 10,000 cps; in the range of about 10 cps to about 1,000 cps; and in the range of about 50 cps to about 250 cps.

In a specific embodiment, the composition may include one or more carbohydrates, including fructose and maltodextrin, water and an active drug.

In a specific embodiment, the methods of the present invention may utilize specific compositions comprising maltodextrin, fructose, water, sodium citrate, potassium citrate, natural or artificial flavors, sodium benzoate and an active drug.

In another specific embodiment, the composition may comprise fructose, maltodextrin, water, sodium citrate, calcium carbonate, sodium benzoate, potassium sorbate, citric acid, pectin, vitamin c and an active drug.

In another specific embodiment, the composition may comprise brown rice syrup, natural flavors, sea salt, potassium citrate, citric acid, magnesium oxide and an active drug.

In another specific embodiment, the composition may comprise fructose, maltodextrin, sodium chloride, sodium citrate, potassium chloride, citric acid, sodium benzoate, potassium sorbate, natural flavor and an active drug.

In another specific embodiment, specific embodiment, the methods of the present invention may utilize compositions comprising by weight about 60% maltodextrin. about 27% water, about 10% fructose, 0.05% sodium benzoate, 1.3% sodium citrate, 0.4% potassium citrate, 0.3% natural and/or artificial flavors, 0.05% potassium sorbate and the appropriate amount, of dosage of the active drug.

In another specific embodiment, specific embodiment, the methods of the present invention may utilize compositions comprising by weight about 68% maltodextrin, about 19% water, about 9% fructose, 0.05% sodium benzoate, 1.3% sodium citrate, 0.4% potassium citrate, 0.3% natural and/or artificial flavors, 0.05% potassium sorbate and the appropriate amount of dosage of the active drug.

The gel dosage forms or compositions may be prepared using methods known to those having ordinary skill in the art. For example, the ingredients may be mixed and heated to about 160 to about 180° F. See U.S. Patent Publication 2005/0095271, which is expressly incorporated by reference herein.

A method of commercial manufacturing is in a steam jacketed kettle, however, there are a number of alternate manufacturing processes that could be used with similar results. Manufacturing processes may be in large industrial or small batches and may exclude certain ingredients. In one example, the manufacturer can prepare the gel dosage form without adding the active drug. Hence, the customer such as a nursing home, may have the convenience of buying the gel dosage form in bulk and then personally adding the active drug necessary for each particular patient. Hence, the provided drug-free gel dosage form allows a facility such as a nursing home to satisfy the various needs of multiple patients in a convenient matter. In another example, the manufacturer can prepare the gel dosage form in bulk that also includes the active drug. Such a product would be advantageous for tailoring to a specific patient or to a drug that is commonly used by the population, such as heart medication with elderly patients. In another example, the manufacturer may prepare the gel dosage form that comprises the active drug and make it available per unit dose. Such an option would be the most convenient for a nursing home to administer to a particular patient. Moreover, the availability of providing it per unit dose allows the manufacturer to take advantage of packaging that would provide convenience for the administration of the gel. Also, when the manufacturer adds the drug, the manufacture can provide more accurate quality control that may not be available to a nursing home or individual caregiver to ensure that the active drug is homogenously mixed with the gel.

One method for the manufacturing and performing quality control is as follows: Mix all of the water with the sodium benzoate and potassium sorbate placed in a Unimix™ brand mixer and mix for at least 10 minutes or until all sodium benzoate and potassium sorbate is dissolved. Add citric acid and color and mix under a moderate speed for at least 10 minutes. Add maltodextrin and mix under moderate speed for at least 30 minutes to ensure a substantially uniform semisolid gel form is obtained. Add fructose and mix under moderate speed for at least 30 minutes to ensure a uniform mixture. Add flavor and mix for at least 10 minutes. Add the active drug in the final step and mix at a moderate speed for at least 1 hour to ensure a homogenous mixture of the active drug within the gel dosage form. Heat to about 120 degrees Fahrenheit and perform quality control to test for a homogenous mixture of the active drug.

To test for a homogenous mixture of the active drug in the gel dosage form, two samples are obtained. This test may be done using a 225 ml container which holds 200 mL volume of prepared gel. A 5 or 10 mL polypropylene syringe with a 12 or 14 gauge 6 inch needle is used and the needle is marked such that it will retrieve a sample from the bottom of the gel and from the top of the gel. For each sample, 5 mL is drawn.

For the top sample, the needle is placed ½ inch into the top of the gel and 5 mL is drawn. The needle is removed from the syringe, the plunger is pulled back to allow for headspace, and 3 mL of the sample is discarded. The end of the syringe is capped. For bottom sampling, another 5 or 10 mL polypropylene syringe with a 12 or 14 gauge, 6 inch needle is used. The needle is placed all the way to the bottom of the container and 5 mL is drawn. The needle is removed, the plunger is pulled back to allow for headspace, and 3 mL of the sample is discarded. The end of syringe is capped. A known weight from the 2 mL of the sample is analyzed for drug content. A sample of the invention is tested for maintaining the medicament in a homogenous mixture so that the top and bottom medicament concentrations are within +/−0.5%. After it has been established that the active drug is homogenously mixed with the gel, the batch may be package as desired. Further testing on the batch may performed by keeping the gel at 40 degree C. for at least about 90 days without shaking at any point during the test period.

The contents may be packaged per unit dose and the packaging allows for various ranges in serving size. In a specific embodiment of the invention, the methods may utilize compositions in the range of about 1 g to about 48 g in serving size. In a specific embodiment of the invention, the methods may utilize compositions in the amount of about 32 g in serving size. Specifically, the serving size or dosage unit may allow for available dosages that are too large for the pill or filmstrip dosage forms. The contents may also be packaged per unit volume. In a specific embodiment, the methods may utilize compositions in the amount of about 1 mL to about 100 mL. In a specific embodiment, the volume of the dosage unit may be about 1 mL to about 10 mL. In a specific embodiment, the volume of the dosage unit may be about 2 mL to 5 mL. In a specific embodiment, the volume of the dosage unit may be about 5 mL.

The methods of the present invention may utilize compositions with various packaging. For example, the composition may be packaged in a laminated foil pouch. The laminated foil pouch may have easy access to the contents by having a tear-off top. There are several alternate packaging alternatives that are suitable for holding and storing the composition including but not limited to: a bottle, a can, a sealed cup, a box, a syringe, a foil or plastic pouch and a tube, all made of a variety of film materials. There are also several alternatives for providing the patient easy access to the contents within the packaging, which may include, but are not limited to, a tear-off top, a means for a straw to be inserted, a flip-up top, and a screw-off top. The contents may also be administered to the patient by various means. For example, the contents may also be administered to the patient by a straw. The contents may be administered by the patient sucking from an intended opening of the packaging.

The methods may also utilize packaging that would be suitable and convenient for a nurse or the like to dole out the present invention to one or more patients. For example, the packaging may be designed to fit in a nursing home medication cart. In another specific example, the packaging may be designed to fit in a patient's individual medication tray.

Another embodiment would be the use of various forms of packaging or methods to administer the drug to a patient so that only a minimal or insignificant amount of gel residue has not been consumed. For example, the packaging may be clear or optically transparent so that the nurse or caregiver has visible confirmation that all the contents have been consumed. In another embodiment, the methods may utilize packaging in a tube or pouch so that the contents may be easily removed by deformation of the packaging. An important factor would be the interior film or material that contains the gel dosage form. For example, the gel dosage form could be contained within a non-adherent film or a film that has a high slip rate. Therefore, the contents will be easily removed by the patient or caregiver with a minimal and insignificant amount of gel residue. In a specific embodiment, the methods of the invention may utilize packaging that allows for over 99% of the contents to be consumed by the patient. In another embodiment, flexible polyvinyl chloride (PVC). polyolefins and polyolefin alloys may be used as the interior film. In a specific embodiment, an elastic material with a minimal adhesion polymer blend for fabricating films may be used. See, for example, U.S. Pat. No. 6,969,483. The containers may therefore be flowable material, for example, an I.V. pouch. Hence, the material when in the form of a container, allows the sample, either liquid or solid, to flow by the force of gravity.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLE 1

A composition of the following formulation was prepared in a gel form by standard methods known to those skilled in the art:

| | | |
|---|---|---|
| maltodextrin | 22 | g |
| fructose | 3 | g |
| water | 6 | g |
| sodium benzoate | 16 | mg |
| potassium sorbate | 16 | mg |
| natural/artificial flavors | 100 | mg |
| metoprolol | 50 | mg |
| citric acid | 416 | mg |
| sodium citrate | 416 | mg |

### EXAMPLE 2

A composition of the following formulation was prepared in a tablet form by standard methods known to those skilled in the art:

| | | |
|---|---|---|
| maltodextrin | 22 | g |
| fructose | 3 | g |
| water | 6 | g |
| sodium benzoate | 16 | mg |
| potassium sorbate | 16 | mg |
| natural/artificial flavors | 100 | mg |
| metoprolol | 50 | mg |
| citric acid | 416 | mg |
| sodium citrate | 416 | mg |

### EXAMPLE 3

A study is undertaken to evaluate the relative effectiveness of the compositions of the present invention in the treatment of patients in numerous dosage forms. The objective of the study is to determine whether oral administration of the compositions in a gel dosage form results in a comparable improvement of the symptoms of cardiovascular disease relative to the oral administration of the compositions in a tablet dosage form.

A study is conducted over a three-month period. A total of 120 subjects, aged 65-80 years, are chosen for the study. The 120 subjects chosen for the study are separated into two separate groups of 60. The characteristics of the symptoms for high blood pressure between the two groups are comparable. In a first group comprising men and women, each subject is administered one dosage form of the composition as described in Example 1 once a day. In a second group comprising men and women, each subject is administered one dosage form of the composition as described in Example 2 once a day. The second group, therefore, administers a medication that is similar in all respects to the administered composition in Example 1 except for the dosage form being a tablet. The patient's blood pressure is examined once a week after initiating the treatment.

(SAS Institute Inc., Cary. N.C.). An alpha level of 0.05 is used in all statistical tests.

The assessment of the lowering of blood pressure is conducted for each subject group. The data is evaluated using multiple linear regression analysis and a standard t-test. In each analysis, the baseline value of the outcome variable is included in the model as a covariant. Treatment by covarient interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at each week, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the comparable efficacy of the composition of the present invention in treating the symptoms of cardiovascular disease relative to other currently available dosage forms. Regarding potential adverse effects of taking the medication, if there are no significant differences between the two therapeutic groups, this study will demonstrate that the administration of the composition of the present invention is effective at treating symptoms of cardiovascular disease in addition to being well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

What is claimed is:

1. A method for facilitated oral administration of an active drug to a patient comprising administering said active drug in a gel form to said patient, wherein said gel form comprises one or more gelling agents and one or more active drugs, and said gel form comprises a homogenous mixture of said active drug.

2. The method of claim 1, wherein said patient has difficulty in swallowing pills.

3. The method of claim 2, wherein said patient is selected from one or more of the group consisting of an elderly patient, a child and an infant.

4. The method of claim 1, wherein said gelling agent comprises a carbohydrate.

5. The method of claim 4, wherein said carbohydrate is selected from one or more of the group consisting of aldohexoses, disaccharides, polysaccharides, ketohexoses, glucose, glucose polymers, dextrose, maltose, maltodextrins, maltotriose, lactose, galactose, sucrose, corn syrup, high fructose corn syrup, honey, maple syrup, molasses, brown rice syrup, beet sugar, cane sugar, sucanat, arabinose, ribose, xylose, fructose, levulose, psicose, sorbose, tagatose, sorbitol and combinations thereof.

6. The method of claim 4, wherein said carbohydrate comprises maltodextrin and fructose.

7. The method of claim 5, wherein said carbohydrate comprises brown rice syrup.

8. The composition of claim 1, wherein said gelling agent is selected from one of more of the group consisting of protein, amino acids, pectin, agar, arabic gum, xanthan gum, tragacanth gum, karaya gum, ghatti gum, guar gum, gellan gum, locust bean gum, alginic acid, a pharmaceutically acceptable alginate salt, carrageenan, gelatin, dextrin, starches, corn starch, rice starch, wheat starch, potato starch, pueraria starch, tapioca starch, carboxymethyl starch, hydroxypropyl starch, hydroxyethyl starch, chemically cross-linked starch, celluloses, hydroxypropylmethyl cellulose, carboxymethyl cellulose, methyl cellulose, methyl ethyl cellulose, hydroxypropyl cellulose, crystalline cellulose, polyvinyl alcohol, polyvinylpyrrolidone, polyethylene glycol, mannans, and combinations thereof.

9. The method of claim 1, wherein said active drug is selected from one or more of the group consisting of an antacid, drugs to treat peptic ulcer disease, antiparkinsons drugs, antidepressants, phosphate binders, anti-psychotics, antiepileptic drugs, anti-hypertensives, cardiovascular drugs, diuretics, Ace inhibitors, Beta blockers, Angiotensin II antagonists, HMG-CoA reductase inhibitors, antiarrhythmic drugs, hormonal, endocrine or drugs for the thyroid, hormones of the posterior pituitary. antidiarreheals, stool softeners, laxatives, drugs for treatment of urinary incontinence, analgesics/antipyretics, drugs used in pediatrics and antibacterials.

10. The method of claim 1, wherein the composition also comprises vitamins, nutrients and/or minerals selected from one of more of the group consisting, of vitamin A, vitamin D, vitamin K, vitamin E, vitamin C, vitamin B₁, vitamin B₂, vitamin B₃, vitamin B₅, vitamin B₆, vitamin B₇, vitamin B₉ and vitamin B₁₂, pyridoxal, pyridoxamine, pantothenic acid, folic acid, sodium, potassium, chloride, magnesium, manganese, and Omega-3 fatty acids.

11. The method of claim 1, wherein said composition comprises a natural or artificial flavor selected from one or more of the group consisting of the flavors apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

12. The method of claim 6, wherein said composition comprises by weight a range of about 90% to about 30% maltodextrin.

13. The method of claim 6, wherein said composition comprises by weight a range of about 50% to about 5% fructose.

14. The method of claim 6, wherein said carbohydrates is the range of about 85% to about 70% carbohydrates from maltodextrin and about 30% to about 15% carbohydrates from fructose.

15. The method of claim 1, wherein said composition comprises maltodextrin, fructose, water, sodium citrate, potassium citrate, natural or artificial flavors and sodium benzoate.

16. The method of claim 1, wherein said composition comprises brown rice syrup, natural flavors, sea salt, potassium citrate, citric acid, and magnesium oxide.

17. The method of claim 1, wherein said composition is packaged per unit dose.

18. The method of claim 1, wherein said composition is in the serving size range of about 1 g to about 48 g.

19. The method of claim 17, wherein said composition is in the serving volume of about 1 mL to about 10 mL.

20. The method of claim 17, wherein said packaging is selected from one or more of the group consisting of a bottle, a can, a sealed cup, a box, a syringe, a foil or plastic pouch and a tube.

21. The method of claim 17, wherein said packaging is selected from one or more of the group consisting of a tear-off top, a means for a straw to be inserted, a flip-up top, and a screw-off top.

22. The method of claim 17, wherein said packaging makes available dosages that are too large for pill or filmstrip dosage forms.

23. The method of claim 1, wherein said composition is substantially free of another added active drug.

24. The method of claim 1, wherein said composition is co-administered with another active drug.

25. The method of claim 1, wherein said composition is substantially free of other added active ingredients.
